(19) United States
(12) Patent Application Publication
(10) Pub. No.: US 2018/0078283 A1
(43) Pub. Date: Mar. 22, 2018

(54) Vacuum-Assisted Vaginal Positioning Device

(71) Applicant: Boston Scientific Scimed, Inc., Maple Grove, MN (US)

(72) Inventors: Peter J. PEREIRA, Mendon, MA (US); Joseph GORDON, Mansfield, MA (US); Faith DAVID-HEGERICH, Holliston, MA (US)

(21) Appl. No.: 15/702,478

(22) Filed: Sep. 12, 2017

Related U.S. Application Data
(60) Provisional application No. 62/396,723, filed on Sep. 19, 2016.

(51) Int. Cl. A61B 17/42; A61B 17/04; A61B 17/34; A61M 29/00; A61M 29/02

(52) U.S. Cl. CPC A61B 17/42; A61B 17/0482; A61B 17/3423; A61F 2/0063; A61M 29/00; A61M 29/02; A61B 17/3468

(57) ABSTRACT

A medical device for tissue manipulation within a body cavity includes a shaft having a lumen extending therethrough from a proximal end to a distal end and a first channel extending within the lumen, an expandable end-effector extending from a proximal end to a distal end, and an expansion mechanism. The end-effector includes a first portion and a second portion and is movable from a first configuration in which the first and second portions are adjacent to one another to a second configuration in which the first and second portions are separated along an axis perpendicular to a longitudinal axis of the shaft. The expansion mechanism is positioned between the first and second portions and expandable to move the end effector from the first configuration to the second configuration.

### PRIORITY CLAIM

The present disclosure claims priority to U.S. Provisional Patent Application Ser. No. 62/396,723 filed Sep. 19, 2016; the disclosure of which is incorporated herewith by reference.

### BACKGROUND

Vaginal prolapse occurs when the bladder, uterus, and/or bowel protrude into the vagina, typically due to loss of natural support for the pelvic organs and the vaginal vault. Vaginal prolapse occurs most often in women who have undergone a prior hysterectomy. However, prolapse may also occur in women who have not had a hysterectomy. Sacrocolpopexy is a surgical technique for repairing pelvic organ prolapse, specifically apical or vaginal prolapse. In treating vaginal prolapse, support of the pelvic organs is generally achieved by attaching a piece of material, such as a mesh, usually from the top and back of the vagina to a ligament of the lower backbone. There is need for a vaginal manipulation device to aid in the attachment of mesh to the vaginal wall in sacrocolpopexy procedures.

### SUMMARY

The present disclosure relates to tissue manipulation device comprising a shaft having a lumen extending therethrough from a proximal end to a distal end and a first channel extending within the lumen, an expandable end-effector extending from a proximal end to a distal end and including a first portion and a second portion, the end-effector being movable from a first configuration in which the first and second portions are adjacent to one another to a second configuration in which the first and second portions are separated along an axis perpendicular to a longitudinal axis of the shaft, and an expansion mechanism positioned between the first and second portions and expandable to move the end effector from the first configuration to the second configuration.

In an embodiment, the expansion mechanism is a balloon bladder.

In an embodiment, fluid is supplied through the first channel to a cavity in the balloon bladder to inflate the balloon bladder.

In an embodiment, the expansion mechanism is a ratcheting mechanism.

In an embodiment, the first and second portions comprise a plurality of vacuum ports configured to pull a target tissue closer to the surface of the device.

In an embodiment, the shaft further comprises at least a second channel configured to provide a vacuum force to the vacuum ports.

In an embodiment, the first and second portions comprise a plurality of raised areas.

In an embodiment, the end-effector is in the form of a paddle.

The present disclosure also relates to tissue manipulation device comprising a shaft extending longitudinally from a proximal end to a distal end, an expandable member coupled to a distal end of the shaft, the expandable member including first and second portions extending from a proximal end of the expandable member to a distal end of the expandable member, and a spreading mechanism configured to move the first and second portions between a first configuration in which the first and second portions are adjacent to one another and a second configuration in which the first and second portions are spread away from one another.

In an embodiment, the shaft includes a lumen extending therethrough and a first channel extending within the lumen.

In an embodiment, the spreading mechanism is a balloon bladder.

In an embodiment, fluid is supplied through the first channel to a cavity in the balloon bladder to inflate the balloon bladder.

In an embodiment, the first and second portions comprise a plurality of vacuum ports configured to pull a target tissue closer to the surface of the device.

In an embodiment, the shaft further comprises at least a second channel configured to provide a vacuum force to the vacuum ports.

In an embodiment, the first and second portions comprise a plurality of raised areas.

The present disclosure also relates to a method for manipulating tissue within a body cavity comprising introducing a medical device into the body cavity. The medical device comprises a shaft having a lumen extending therethrough from a proximal end to a distal end and a first channel extending within the lumen, an expandable end-effector including a first portion and a second portion, the end-effector being movable between a first configuration in which the first and second portions are adjacent to one another and a second configuration in which the first and second portions are separated along an axis perpendicular to a longitudinal axis of the shaft, and an expansion mechanism positioned between the first and second portions. The method also includes expanding the end-effector from the first configuration to the second configuration to fit a profile of the body cavity, and suctioning a target tissue to the end-effector via a plurality of vacuum ports located on the first and second portions.

In an embodiment, the expansion mechanism is a balloon bladder.

In an embodiment, fluid is supplied through the first channel to a cavity in the balloon bladder to inflate the balloon bladder.

In an embodiment, the shaft further comprises at least a second channel configured to provide a vacuum force to the vacuum ports.

In an embodiment, the first and second portions comprise a plurality of raised areas.

### BRIEF DESCRIPTION

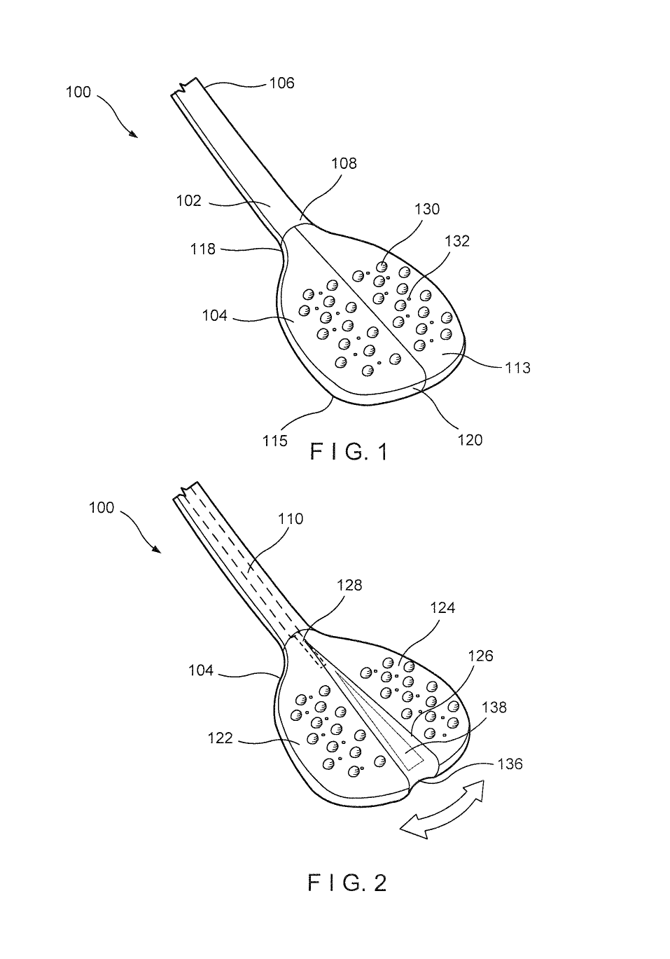

FIG. 1 is a schematic view of an exemplary tissue manipulation device according to a first exemplary embodiment of the present disclosure;

FIG. 2 shows another schematic view of the tissue manipulation device of FIG. 1 in an expanded configuration;

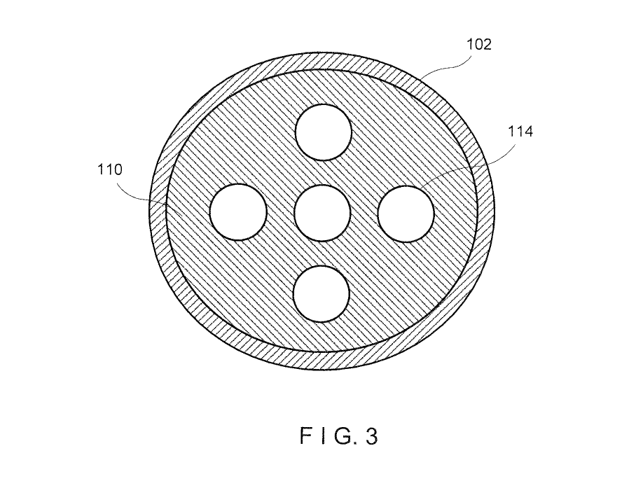

FIG. 3 depicts a cross-section view of the shaft of the manipulation device of FIG. 1;

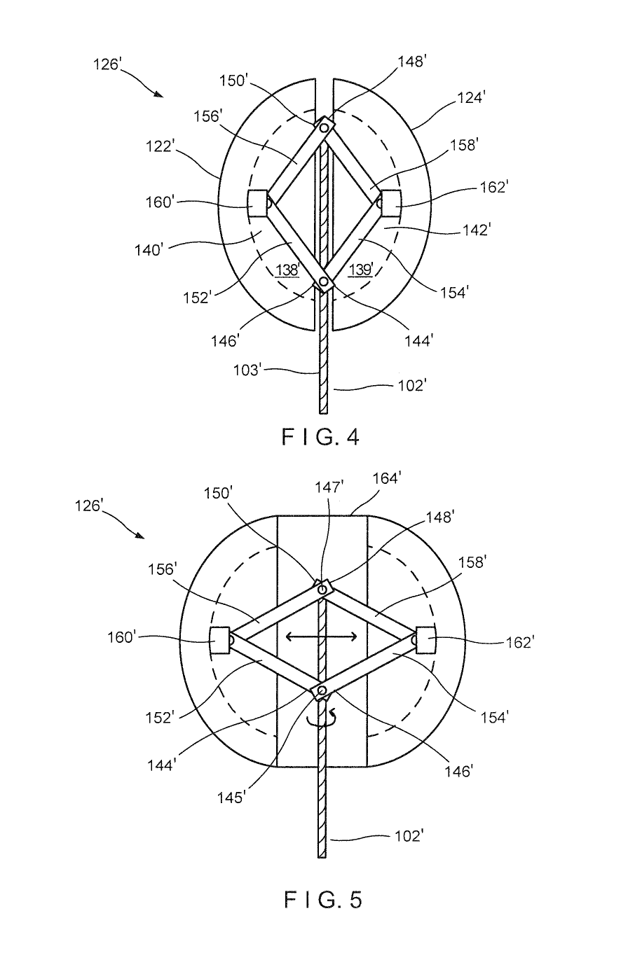

FIG. 4 depicts a partial cross-sectional view of an exemplary tissue manipulation device according to a second exemplary embodiment of the present disclosure;

FIG. 5 depicts another partial cross-sectional view of the exemplary tissue manipulation device of FIG. 4;

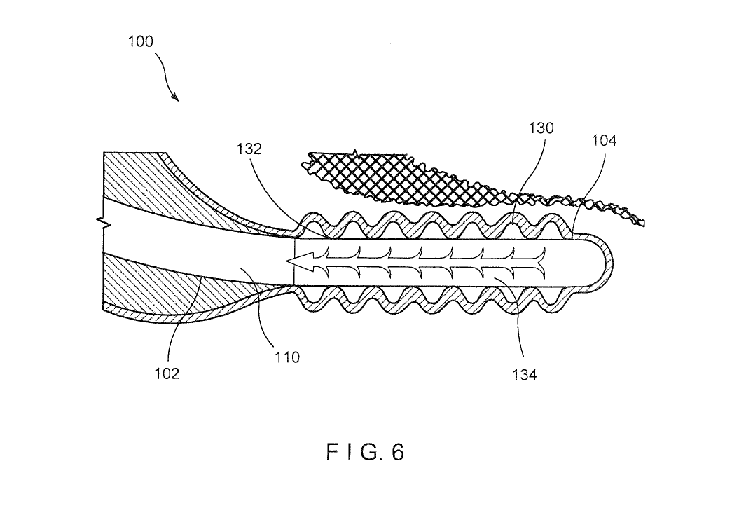

FIG. 6 is an exemplary method of using the manipulation device of FIG. 1 to suture mesh to a vaginal wall;

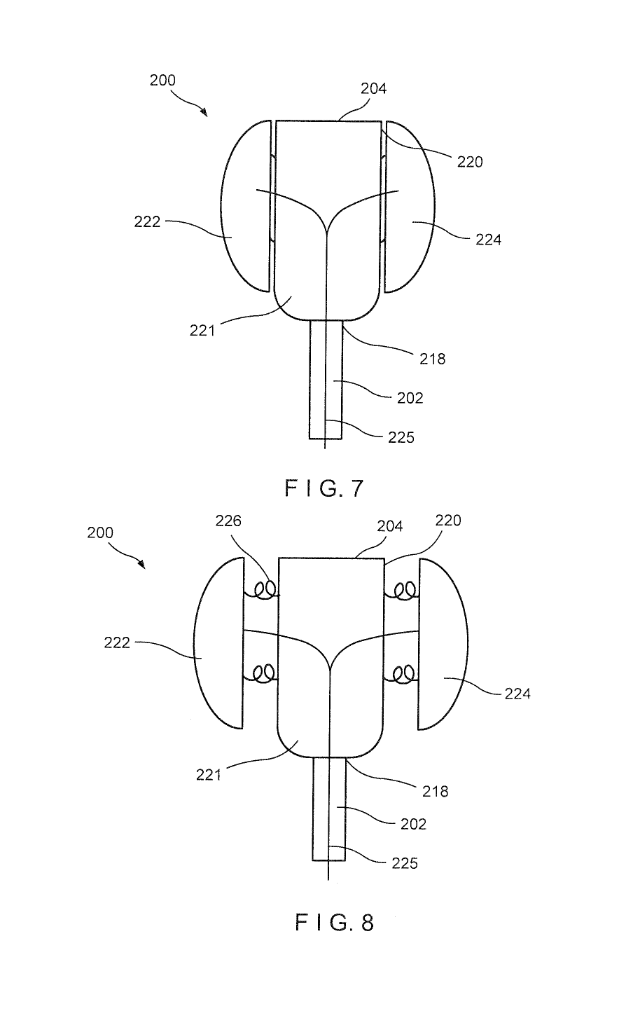

FIG. 7 depicts an exemplary tissue manipulation device according to a third exemplary embodiment of the present disclosure; and

FIG. 8 depicts another side-view of the exemplary tissue manipulation device of FIG. 7.

### DETAILED DESCRIPTION

The present disclosure may be further understood with reference to the appended drawings and the following description, wherein like elements are referred to with the same reference numerals. It should be noted that the terms “proximal” and “distal,” as used herein, are intended to refer to a direction toward (proximal) and away from (distal) a user of the device (e.g. physician).

The present disclosure relates to medical devices and methods for positioning and attaching mesh to the vaginal wall and, more particularly, to a tissue manipulation device for positioning and suturing mesh to the vaginal wall during sacrocolpopexy procedures. The manipulation device includes a shaft having a proximal end, a distal end, and a lumen extending between the proximal and distal ends. One or more working channels extend within the lumen of the shaft from a proximal end of the shaft to its distal end. For the suturing process, an end effector is disposed at the distal end of the shaft to aid in manipulation of the vaginal wall. The end-effector is configured as an expandable paddle-shaped member, including two paddle halves, that moves between two configurations—expanded and insertion/collapsed. The expandable member remains collapsed during insertion into the vagina and during retrieval of the device. Once deployed to a desired position within the target body cavity, the expandable member may increase in width by separation or spreading outward of the two paddle halves. The expanded state of the paddle allows the device to better fit the profile of the vagina to assist in proper mesh fixation as will be described below.

The described embodiments of the present disclosure relate to tissue manipulation for sacrocolpopexy procedures. However, it will be understood by those skilled in the art that the medical device may be employed for other medical procedures.

FIGS. 1-3 illustrate an exemplary tissue manipulation device 100 according to an embodiment of the present disclosure. The device 100 includes a shaft 102 and an expandable paddle member 104 coupled to a distal end of the shaft 102 to move the device from an insertion configuration, as shown in FIG. 1, to an expanded configuration, as shown in FIG. 2, to fit to the profile of the vagina. The shaft 102 extends longitudinally from a proximal end 106 to a distal end 108 and includes a lumen 110 extending between the proximal and distal ends 106, 108. The device 100 also includes one or more working channels 114 running within the lumen 110 of the shaft 102 from its proximal end 106 to its distal end 108. The manipulation device 100 may further include a handle (not shown) coupled to a proximal end 106 of the shaft 102. The manipulation device 100 may also include one or more ports on the shaft 102 or handle (not shown) to connect external equipment to the working channels 114 of the shaft 102. The manipulation device may also be adapted to be connected to a fluid reservoir (not shown) that stores fluid, such as saline.

The shaft 102 may be substantially tubular and it may be made of any suitable biocompatible material such as polyurethane, plastic, or any other such material. Other suitable cross-sectional shapes such as elliptical, oval, polygonal, or irregular may also be contemplated. The shaft 102 is preferably rigid along its entire length. However, the shaft 102 may be flexible along its entire length or adapted for flexure along portions of its length. Alternatively, the shaft's distal end 108 may be flexible while the remaining proximal portion of the shaft 102 is rigid. Flexibility allows the shaft 102 to maneuver in circuitous vasculature, while rigidity provides the required force to urge the shaft 102 forward.

Operators may insert multiple medical devices through the working channels 114 to perform various procedures simultaneously. For example, to expand the expandable member 104, one of the working channels 114 may allow fluid into and out of the distal end 108 of the shaft 102. A syringe may be connected to one of the working channels 114 to insert fluid into and draw fluid from the expandable member 104. It will be understood that other exemplary tools also may be inserted in the working channels 114 without departing from the scope of the present disclosure.

Furthermore, the handle (not shown) may include one or more ports for inserting tools into the manipulation device 100. The handle (not shown) may further include means to manipulate the distal end 108 of the shaft 102 to facilitate navigation of the device within the body. Such means may include mechanical, electronic, or combination means as are known in other steerable endoscopic devices. Further, the handle (not shown) may include control buttons to actuate the expandable member 104 as would be understood by those skilled in the art.

The expandable member 104 may be, for example, a substantially paddle-shaped outwardly expansible member including a first side 113 and a second side 115 extending from a proximal end 118 of the member 104 to a distal end 120 of the member 104. The exemplary paddle-shaped expandable member 104 is substantially circular/ovoid in shape with substantially flat first and second sides 113, 115 that taper at a proximal portion to join with the shaft 102. The expandable member 104 of this embodiment increases in width from the proximal end 118 to a maximum width at a distal end 120. The rounded edges enable easier and safer access to the vagina while minimizing damage to surrounding tissue. The expandable member 104 is divided into a first half portion 122 and a second half portion 124 which are separable along an axis substantially coincident with a longitudinal axis of the shaft 102 via an expansion mechanism 126 located between the first and second half portions 122, 124, respectively. The expandable member 104 further includes a channel 128 extending from the proximal end 118 at least partially therethrough to the expansion mechanism 126. The channel 128 is sized and shaped to be in open communication with at least one of the working channels 114 of the shaft 102. The expandable member 104 may be formed of Acrylonitrile Butadiene Styrene (ABS) or any other suitable biocompatible material. In an exemplary embodiment, the expandable member 104 is sized and shaped to be inserted into and fit a profile of the vagina. The length of the expandable member 104 is approximately 6.5 centimeters (cm) to 12.5 cm and, more specifically, 8.5 centimeters (cm). The width of the expandable member 103 in a collapsed configuration is approximately 3.5 cm while the width of the expandable member 104 in an expanded configuration is up to approximately 7 cm. In other words, the expansion mechanism 126 may increase the width of the expandable member 104 by up to 3.5 cm.

First and second sides 113, 115 include raised areas or bumps 130 distributed thereabout to help elevate specific areas of the vaginal tissue to aid in suturing a mesh to the vaginal wall. Bumps 130 may also be used as visual landmarks to ensure the mesh is placed evenly along the vaginal tissue. In an exemplary embodiment, bumps 130 are substantially circular in shape, although other shapes and configurations may be contemplated. Bumps 130 are raised between 3 and 5 mm from the surface of first and second sides 113, 115 and are spaced between 0.5 and 1 cm apart from one another. In the embodiment of FIGS. 1-2, bumps 130 are randomly distributed about the surfaces of the first and second sides 113, 115. However, it is understood that bumps 130 may be distributed in any other suitable manner, such as, for example, in a grid configuration. In another exemplary embodiment, bumps 130 may be distributed about the surfaces of the first and second sides 113, 115 such that each of the bumps 130 is spaced a specific distance apart from adjacent bumps 130. In this embodiment the user may use the bumps 130 to identify and measure locations of landmarks within the vaginal wall. For example, the bumps 130 may be used to identify locations of thicker vaginal tissue, which are ideal for suturing of the mesh. In the embodiment shown, the expandable member 104 approximately 28 bumps 130 distributed on each of the first and second sides 113, 115. However, it is understood that any number of bumps 130 may be used.

Expandable member 104 includes a plurality of vacuum ports 132 distributed about the surfaces of one or both of the first and second sides 113, 115. Vacuum ports 132 pull tissue closer to the surface of the expandable member 104 and hold the tissue in place to allow for better positioning of the mesh, facilitating the suturing of the mesh to the vaginal wall, and enabling the user to determine tissue thickness along the vaginal surface. Vacuum ports 132, by holding tissue to the surface of the expandable member 104 also allow the physician to rotate tissue if needed. The vacuum ports 132 are fluidly coupled to a working channel 114 via chambers 134 within each the first and second half portions 118, 120 of the expandable member 104. The chambers 134 extend from a proximal portion to a distal portion of the expandable member 104 so that suction may be applied to tissue adjacent to substantially the entire length of the expandable member 104. Suction is applied through the working channel 114, chambers 134, and vacuum ports 132 via, for example, an external suction mechanism (not shown). The suction mechanism may be mechanical, electronic or a combination thereof as would be understood by those skilled in the art.

To shift between the collapsed and expanded states, the expanding mechanism 126 of the expandable member 104 may be self-expandable or may be expanded by a mechanism actuated by a user. In an exemplary embodiment, first and second member halves 122, 124 are separated from one another by a balloon bladder 136 situated between the two member halves 122, 124 as shown in FIGS. 1-2. The balloon bladder 136 has an internal cavity 138 which may be inflated by providing air, saline, or any other suitable fluid to the cavity 138 under pressure. The balloon bladder 136 may be formed, for example, of silicon, urethane, or any other suitable biocompatible material. The inflating fluid is delivered to the expandable member 104 through a working channel 114 of the shaft 102 and enters the cavity 138 of the balloon bladder 136 via the channel 128. The fluid may be stored in the reservoir (not shown) in which it resides until it is provided to the balloon bladder 136. The reservoir may be a metallic, ceramic, or polymeric container that can contain an appropriate amount of fluid required for the inflation procedure. In addition, the reservoir may be connected to the working channels 114 via one or more ports (not shown) on the shaft 102 or the handle (not shown) of the device 100. Suitable actuators connected to the reservoir may pump the fluid to the working channels 114. Balloon bladder 136 may be filled to the degree necessary to expand the expandable member 104 to fit the profile of the body lumen. That is, the balloon bladder 136 need not be filled completely if the body lumen is only slightly larger than the profile of the collapsed member 104.

To return the expandable member 104 into a collapsed state, a suitable drainage mechanism may be employed to remove and dispose of the fluid after the procedure. If the inflating fluid is saline or some similar liquid, the fluid may be suctioned out by a suction or vacuum mechanism (not shown). The vacuum mechanism may be mechanical, electronic or a combination thereof. In an embodiment, a vacuum mechanism is attachable to the working channel 114 via ports in the handle (not shown). Applying a vacuum through the working channel 114 suctions the inflating fluid from the balloon bladder 136 and out through the channel 128 and working channel 114. In an alternate embodiment, a syringe may be inserted through the lumen 110 of the shaft 102 so that a distal end enters the balloon bladder cavity 138 to draw the fluid out of the balloon bladder 136. A disposable or re-usable container may be connected to the working channels 114 of the shaft 102 via one or more ports on the shaft 102 or the handle (not shown). The drainage mechanism may also employ suitable actuators or pumps to drain or extract the fluid from the working channels 114 to the drainage container.

Other variations of the expansion mechanism 126 may be contemplated. For example, in another exemplary embodiment, as shown in FIGS. 4-5, the expansion mechanism 126′ includes first and second halves 122′, 124′ that may be moved apart from one another as described below. The expansion mechanism 126′ includes a first portion 140′ and second portion 142′ which extend from proximal ends 144′, 146′, respectively, to distal ends 148′, 150′, respectively. The first portion 140′ includes a proximal portion 152′ and a distal portion 156′ coupled to one another at a hinge 160′. Similarly, the second portion 142′ includes a proximal portion 154′ and a distal portion 158′ coupled to one another at a hinge 162′. The first and second portions 140′, 142′ are pivotally coupled to one another at proximal and distal ends 144′, 146′, 148′, 150′ via pins 145′ and 147′, respectively or in any other suitable manner. One of the proximal ends 144′, 146′ includes a threaded bore (not shown) through which the shaft 102′ is inserted. The shaft 102′ includes an external threading 103′ along at least a portion of its length which cooperates with the internal threading of the bore so that, as the shaft 102′ is rotated relative to the bore in a first direction, the proximal ends 144′, 146′ are moved along the shaft 102′ toward the distal ends 148′. 150′ to laterally expand the expansion mechanism 126′. Rotation of the shaft 102′ in a second direction opposite the first direction moves the proximal ends 144′, 146 away from the distal ends 148′, 150′ to contract the expansion mechanism 126′ laterally. The first and second halves 122′, 124′ each include an internal cavity 138′, 139′ open to medial surfaces thereof to house the expansion mechanism 126′. The first and second portions 140′, 142′ may be coupled to first and second halves 122′, 124′, respectively, in any suitable manner. In an exemplary embodiment, outer lateral surfaces of first and second hinges 160′, 162′ are coupled to inner surfaces of first and second cavities 138′, 139′, respectively. Rotation of the shaft 102′ in the first direction moves the proximal ends 144′, 146′ of first and second portions 140′, 142′ distally along the shaft 102′ so that the angle between proximal portions 152′, 154′ and distal portions 156′, 158′ decreases, causing first and second halves 122′, 124′ to spread apart. In an insertion configuration, the angle between proximal portions 152′, 154′ and distal portions 156′, 158′ of first and second portions 140′, 142′ may be approximately 135-180 degrees. In a laterally expanded configuration, the angle between proximal portions 152′, 154′ and distal portions 156′. 158′ of first and second portions 140′, 142′ may be decreased to an angle of, for example, 25-90 degrees, depending on the amount of expansion needed. In an exemplary embodiment, expandable member 104′ may include a connecting member 164′ extending from a medial surface of the first half 122′ to a medial surface of the second half 124′ on each of first and second sides 113′, 115′ to protect the body tissues from contact with the expansion mechanism 126′ and vice versa. The connecting member 164′ may be formed of, for example, silicon, urethane, or any other suitable biocompatible material.

FIG. 6 illustrates an exemplary method for manipulating tissue using the manipulation device 100. Specifically, the manipulation device 100 manipulates wall tissue within the vaginal canal to facilitate attaching a mesh in a sacrocolpopexy procedure. It should be understood that the manipulation device 100 may be used to manipulate vaginal tissue for other procedures or to manipulate tissue in other body cavities without departing from the scope of the present disclosure.

The method includes advancing the shaft 102 into the vaginal canal through the vaginal opening. The proximal end of the shaft 102 includes a handle and/or controller (not shown), which, during use, remain outside the body accessible to the user. The shaft 102 carries the expandable member 104 coupled to a distal end thereof in its collapsed form. Once the shaft 102 has been advanced into the vaginal canal to a desired position with the expansion mechanism 126 positioned adjacent to target tissue to be manipulated, the expansion mechanism is actuated via an actuator on the handle to spread the two member halves 122, 124 apart from one another, increasing the width of the expandable member 104. In an exemplary embodiment, the expansion mechanism is the balloon bladder mechanism 126. Fluid (e.g., sterile saline) is inserted into the cavity 138 of the balloon bladder 126 through a working channel 114 under pressure to expand the balloon bladder 126 and push the halves 122, 124 away from each other. The fluid injected through the channel 114 remains within the cavity 138 for a desired period of time, which may depend on the extend of the sacrocolpopexy procedure required after which it is evacuated to return the balloon bladder 126 to its non-expanded state returning the halves 122, 124 to their insertion configuration.

When the expandable member 104 has been spread to better fit the profile of the vagina, the suction mechanism is actuated to apply negative pressure to the ports 132 pulling adjacent tissue against the surface of the expandable member 104. While the tissue is held against the expandable member 104, raised areas/bumps 130 on the first and second sides 114, 115 help elevate selected areas of vaginal tissue to aid in the suturing of the mesh to the vaginal wall. The raised bumps 130 may also be used as visual land marks to ensure the mesh is placed evenly along the vaginal tissue. The bumps 130 also facilitate determining a thickness of tissue along the vaginal wall to aid in deciding where to suture the mesh to the vaginal wall. During this process, the physician may choose to introduce other tools through the channels 114 of the shaft 102 as would be understood by those skilled in the art. It is further noted that any suitably sized and shaped device may be introduced through the channel of the device 100 without deviating from the scope of the invention.

Once the mesh suturing procedure has been completed, the negative pressure applied through the vacuum ports 132 is stopped to release the vaginal wall from the surfaces of the expandable member 104. Subsequently, the expansion mechanism 126 is collapsed by expelling the fluid or air from the cavity 138 through a fluid outlet channel 114 to the drainage container. Finally, the sheath 102 and expandable member 104 are retracted from the body cavity.

FIGS. 7-8 illustrate an exemplary tissue manipulation device 200 according to a second embodiment of the present disclosure. Expandable device 200 is configured and structured in the same manner as expandable device 100 except as specified hereafter. Device 200 includes a shaft 202 and an expandable member 204 coupled to a distal end of the shaft 202 to move the device from an insertion configuration, as shown in FIG. 5, to an expanded configuration, as shown in FIG. 6, to fit to the profile of the vagina. As with expandable member 104, expandable member 204 may be, for example, a substantially paddle-shaped outwardly expansible member including a first side and a second side extending from a proximal end 218 to a distal end 220. However, in this embodiment, expandable member 204 is divided into a first body portion 221 and two moveable outer portions 222, 224 which are separable from the body portion 221 via expansion elements 226.

In this exemplary embodiment, expansion elements 226 are configured as a plurality of spring elements 226. FIG. 6 shows four spring elements 226, two spring elements 226 extending between the body portion 221 and outer portion 222 and two more spring elements 226 extending between body portion 221 and outer portion 224. It is noted that although four spring elements 226 are used in the embodiment, any number of spring elements may be contemplated. Spring elements may be formed of stainless steel, Nitinol, or any other suitable material. In an insertion configuration, the spring elements are compressed with outer portions 222, 224 abutting the body portion 221. As would be understood by those skilled in the art, the body portion 221 may be maintained in the insertion configuration by a pull wire/suture 225 or any other similar mechanism, which, until released, holds the outer portions 222, 224 against the body portion 221 maintaining the spring elements 226 in a compressed state. Pull wire 225 extends through shaft 202 and the body 221 of the expandable member 204 and is coupled to each of the two moveable outer portions 222, 224. The pull wire 225 may be coupled to the outer portions 222, 224 by any known method such as, for example, welding or adhesive. When the pull wire 225 is released, the spring elements 226 are freed to push the outer portions 222, 224 away from one another to expand the body portion 221 outward. An actuator which, when operated, releases the pull wire mechanism may be provided on the handle (not shown) allowing operators to easily expand release the outer portions 222, 224 as required. Spring elements 226 may be coupled to outer portions 222, 224 and body portion 221 in any suitable manner such as, for example, welding or adhering.

To return the expandable member 204 to the compressed insertion configuration, pull wires (not shown), each of which extends from a proximal end coupled to an actuator to a distal end coupled to a corresponding one of the outer portions 222, 224, may be drawn proximally (e.g., by operating a pull wire actuator on the handle (not shown) until the latch catches again to lock the body portion 221 in the insertion configuration. Pull wires extend through a channel in the expandable member 204 and a working channel 114 of the shaft 202. Pull wires may be coupled to an actuating mechanism within the handle (not shown) or may be actuated manually. When the pull wire is pulled proximally, spring elements 226 are compressed and outer portions 222, 224 are pulled inward to latch, via the latch mechanism, to the body portion 221.

It will be understood by those of skill in the art that various modifications and variations can be made in the structure and methodology of the present invention without departing from the spirit or scope of the invention. Thus, it is intended that the present invention cover the modifications and variations of this invention provided that they come within the scope of the appended claims and their equivalents.

1-15. (canceled)

16. A tissue manipulation device, comprising:
a shaft having a lumen extending therethrough from a proximal end to a distal end and a first channel extending within the lumen;
an expandable end-effector extending from a proximal end to a distal end and including a first portion and a second portion, the end-effector being movable from a first configuration in which the first and second portions are adjacent to one another to a second configuration in which the first and second portions are separated along an axis perpendicular to a longitudinal axis of the shaft; and
an expansion mechanism positioned between the first and second portions and expandable to move the end effector from the first configuration to the second configuration.

17. The device of claim 16, wherein the expansion mechanism is a balloon bladder.

18. The device of claim 17, wherein fluid is supplied through the first channel to a cavity in the balloon bladder to inflate the balloon bladder.

19. The device of claim 16, wherein the expansion mechanism is a ratcheting mechanism.

20. The device of claim 16, wherein the first and second portions comprise a plurality of vacuum ports configured to pull a target tissue closer to the surface of the device.

21. The device of claim 20, wherein the shaft further comprises at least a second channel configured to provide a vacuum force to the vacuum ports.

22. The device of claim 16, wherein the first and second portions comprise a plurality of raised areas.

23. The device of claim 16, wherein the end-effector is in the form of a paddle.

24. A tissue manipulation device, comprising:
a shaft extending longitudinally from a proximal end to a distal end;
an expandable member coupled to a distal end of the shaft, the expandable member including first and second portions extending from a proximal end of the expandable member to a distal end of the expandable member; and
a spreading mechanism configured to move the first and second portions between a first configuration in which the first and second portions are adjacent to one another and a second configuration in which the first and second portions are spread away from one another.

25. The device of claim 24, wherein the shaft includes a lumen extending therethrough and a first channel extending within the lumen.

26. The device of claim 25, wherein the spreading mechanism is a balloon bladder.

27. The device of claim 26, wherein fluid is supplied through the first channel to a cavity in the balloon bladder to inflate the balloon bladder.

28. The device of claim 24, wherein the first and second portions comprise a plurality of vacuum ports configured to pull a target tissue closer to the surface of the device.

29. The device of claim 28, wherein the shaft further comprises at least a second channel configured to provide a vacuum force to the vacuum ports.

30. The device of claim 24, wherein the first and second portions comprise a plurality of raised areas.

31. A method for manipulating tissue within a body cavity, comprising:
introducing a medical device into the body cavity, the medical device comprising:
a shaft having a lumen extending therethrough from a proximal end to a distal end and a first channel extending within the lumen;
an expandable end-effector including a first portion and a second portion, the end-effector being movable between a first configuration in which the first and second portions are adjacent to one another and a second configuration in which the first and second portions are separated along an axis perpendicular to a longitudinal axis of the shaft; and
an expansion mechanism positioned between the first and second portions;
expanding the end-effector from the first configuration to the second configuration to fit a profile of the body cavity; and
suctioning a target tissue to the end-effector via a plurality of vacuum ports located on the first and second portions.

32. The method of claim 31, wherein the expansion mechanism is a balloon bladder.

33. The method of claim 32, wherein fluid is supplied through the first channel to a cavity in the balloon bladder to inflate the balloon bladder.

34. The method of claim 31, wherein the shaft further comprises at least a second channel configured to provide a vacuum force to the vacuum ports.

35. The method of claim 31, wherein the first and second portions comprise a plurality of raised areas.
